SEQUENCE LISTING (12 sequences)

SEQ ID NO: 1 - DNA, 22 bp, Artificial Sequence
  synthetic
gtttactgtcgagcttcgtgat

SEQ ID NO: 2 - DNA, 21 bp, Artificial Sequence
  synthetic
aaggggagcgacagcctgtgt

SEQ ID NO: 3 - DNA, 19 bp, Artificial Sequence
  synthetic
cggaaatgccccatggact

SEQ ID NO: 4 - DNA, 23 bp, Artificial Sequence
  synthetic
tttagtgcaatggaacagtttca

SEQ ID NO: 5 - DNA, 21 bp, Artificial Sequence
  synthetic
atttaaccgttcatcaccctt

SEQ ID NO: 6 - DNA, 21 bp, Artificial Sequence
  synthetic
gacgcttttaagcctgccgtt

SEQ ID NO: 7 - DNA, 24 bp, Artificial Sequence
  synthetic
tcagtcaagatgagagtcttctct

SEQ ID NO: 8 - DNA, 26 bp, Artificial Sequence
  synthetic
caattgtagactttgagtcactgaat

SEQ ID NO: 9 - DNA, 22 bp, Artificial Sequence
  synthetic
gcacatctcaaactccatatca

SEQ ID NO: 10 - DNA, 21 bp, Artificial Sequence
  synthetic
gaaaatgtgttcccaccaacc

SEQ ID NO: 11 - DNA, 22 bp, Artificial Sequence
  synthetic
aaaggtcaggtcatttttgcta

SEQ ID NO: 12 - DNA, 21 bp, Artificial Sequence
  synthetic
atgttgggcttctcgcatctt

(19) United States
(12) Patent Application Publication
(10) Pub. No.: US 2021/0010023 A1
(43) Pub. Date: Jan. 14, 2021

(54) USE OF HAPLOTYPE OF SNP SITE ASSOCIATED WITH HYPOXIA TOLERANCE IN BREEDING OF MEGALOBRAMA AMBLYCEPHALA

(71) Applicant: SHANGHAI OCEAN UNIVERSITY, Shanghai (CN)

(72) Inventors: Shuming ZOU, Shanghai (CN); Dongdong WANG, Shanghai (CN); Guodong ZHENG, Shanghai (CN); Jie CHEN, Shanghai (CN)

(21) Appl. No.: 16/899,540

(22) Filed: Jun. 11, 2020

(30) Foreign Application Priority Data
Jun. 11, 2019 (CN) 201910501085.4

(51) Int. Cl. C12N 15/85; A01K 67/02

(52) U.S. Cl. CPC C12N 15/8509; A01K 2227/40; A01K 67/02

(57) ABSTRACT

A method of breeding a hypoxia-tolerant Megalobrama amblycephala strain, including: (1) screening genes associated with hypoxia tolerance in Megalobrama amblycephala based on transcriptome data; (2) screening SNP sites from the genes associated with hypoxia tolerance; and (3) applying a haplotype of the SNP sites in the molecular marker-assisted breeding of Megalobrama amblycephala to obtain the hypoxia-tolerant Megalobrama amblycephala strain. The genes associated with hypoxia tolerance include Epo, Hif1α, Hif2α, VhI, Hif1an, Vegfaa Egln1a, Egln1b, Egln2 and Egln3, and the haplotype of the SNP sites is T³⁹⁷T⁷¹⁵ of gene Egln2.

### REFERENCE TO AN ELECTRONIC SEQUENCE LISTING

The contents of the electronic sequence listing (Untitled_ST25.txt; Size: 3,000 bytes; and Date of Creation: Aug. 25, 2020) is herein incorporated by reference in its entirety.

### CROSS-REFERENCE TO RELATED APPLICATIONS

This application claims the benefit of priority from Chinese Patent Application No. 201910501085.4, filed on Jun. 11, 2019. The content of the aforementioned application, including any intervening amendments thereto, is incorporated herein by reference in its entirety.

### TECHNICAL FIELD

This application relates to breeding, and more particularly to a use of a haplotype of SNP site associated with hypoxia tolerance in the breeding of Megalobrama amblycephala.

### BACKGROUND

Megalobrama amblycephala named blunt snout bream, pertaining to the genus Megalobrama in the family Cyprinidae of the order Cypriformes, is also commonly known as Wuchang bream. Megalobrama amblycephala is a commercially important herbivorous fish species in the freshwater aquaculture in China with characteristics of delicate flesh quality, high flesh content, wide feeding habit, fast growth and strong disease resistance. In 2018, the total output of Megalobrama amblycephala in China amounted to about 830,000 tons. However, Megalobrama amblycephala is also a hypoxia-sensitive fish species. Megalobrama amblycephala is prone to surfacing and asphyxiating due to the low hypoxia tolerance, and thus it is urgently required to obtain a breed with excellent hypoxia tolerance to promote the development of the Megalobrama amblycephala aquaculture.

### SUMMARY

An object of this application is to provide a use of a haplotype of SNP site associated with hypoxia tolerance in the breeding of Megalobrama amblycephala, which can be used for the molecular marker-assisted breeding of a new Megalobrama amblycephala strain with hypoxia tolerance.

Technical solutions of this application are specifically described as follows.

This application provides a method of breeding a hypoxia-tolerant Megalobrama amblycephala strain, comprising:

(1) screening genes associated with hypoxia tolerance in Megalobrama amblycephala based on transcriptome data;

(2) screening SNP sites from the genes associated with hypoxia tolerance; and

(3) applying a haplotype of the SNP sites in the molecular marker-assisted breeding of Megalobrama amblycephala to obtain the hypoxia-tolerant Megalobrama amblycephala strain.

In an embodiment, the genes associated with hypoxia tolerance comprise Epo, Hif1α, Hif2α, VhI, Hif1an, Vegfaa, Egln1a, Egln1b, Egln2 and Egln3.

In an embodiment, the SNP sites are located at positions 397 and 715 of gene Egln2; and the haplotype of the SNP sites is T³⁹⁷T⁷¹⁵.

In an embodiment, the step (2) comprises:

comparing a sequence of each of the genes associated with hypoxia tolerance with genome sequence of Megalobrama amblycephala to obtain an intron and an exon of each of the genes associated with hypoxia tolerance;

designing a pair of primers within the intron of each of the genes associated with hypoxia tolerance to amplify the corresponding exon; and

comparing a sequence of the amplified product with a sequence of the corresponding exon in genome of Megalobrama amblycephala to screen the SNP sites;

wherein the SNP sites have a polymorphism information content greater than 0.5.

In this application, the diplotype II of gene Egln2 exhibits higher hypoxia tolerance, and thus it can be applied to the molecular marker-assisted breeding of a new hypoxia-tolerant Megalobrama amblycephala strain.

### BRIEF DESCRIPTION OF THE DRAWINGS

FIG. 1 shows the critical oxygen tension of diploid types I and II at which they lose the equilibrium, where different letters indicate that there is statistically significant difference between the diploid type I and diploid type II respectively at 10 10° C., 25° C. and 30° C. (p<0.01); *p<0.01; I: diploid type I strain; II: diploid type II strain.

FIGS. 2A-2H show the optical microstructure of gill lamellas of diploid type I strain and diploid type II strain (temperature: 10±1.0° C.; dissolved oxygen (DO): 2.0 mg/L; scale: 50 μm), where FIG. 2A: diploid type I strain after 4 days of normoxia; FIG. 2B: diploid type I strain after 4 days of hypoxia; FIG. 2C: diploid type I strain after 7 days of hypoxia; FIG. 2D: diploid type I strain after 7 days of normoxic recovery; FIG. 2E: diploid type II strain after 4 days of normoxia; FIG. 2F: diploid type II strain after 4 days of hypoxia; FIG. 2G: diploid type II strain after 7 days of hypoxia; FIG. 2H: diploid type II strain after 7 days of normoxic recovery.

FIGS. 3A-3B respectively show the changes of mass-specific lamellae area and interlamellar cell mass (ILCM) volume in the diploid type I strain and diploid type II strain (temperature: 10±1.0° C.; DO: 2.0 mg/L), where 0 d-H: normoxia; 4 d-H: 4 days of hypoxia; 7 d-H: 7 days of hypoxia; I: diploid type I strain; II: diploid type II strain; *p<0.01; different letters indicate significant difference, p<0.01.

FIGS. 4A-4B respectively show the changes of the red blood cell count and the hemoglobin concentration in the diploid type I strain and diploid type II strain (temperature: 10±1.0° C.; DO: 2.0 mg/L), where 0 d-H: normoxia; 4 d-H: 4 days of hypoxia; 7 d-H: 7 days of hypoxia; I: diploid type I strain; II: diploid type II strain; *p<0.01; different letters indicate significant difference, p<0.01.

### DETAILED DESCRIPTION OF EMBODIMENTS

The disclosure will be described in detail below with reference to the embodiments, but is not limited thereto. Unless otherwise specified, the experiments in the following embodiments are all performed using a conventional method, and the materials and reagents used below are all commercially available.

Prolyl hydroxylase (PHD), as an important oxygen sensor in the regulation of hypoxia-inducible factors, has three kinds of subunits, namely PHD1, PHD2 and PHD3, where the PHD1-encoding gene is also known as Egln2 (Egg laying Deficient Nine-like Protein 2), and these subunits pertain to the non-heme and iron-dependent dioxygenase family which is dependent from oxygen, α-ketoglutaric acid and Fe²⁺, and all can catalyze the hydroxylation of Hif1a (hypoxia-inducible factor 1-alpha), regulating the angiogenesis and erythropoiesis. Currently, related researches have been conducted in mammals such as humans and mice and other animals. It has been reported that the mutation involving the gene Egln1 is closely associated with the evolution of human adaptability to high altitude hypoxic environment. In the Hif signal pathway in response to hypoxia in fish, the gene Egln can degrade the ubiquitinated Hif1a by modification to regulate the oxygen balance in vivo.

Based on the known transcription information associated with hypoxia tolerance in Megalobrama amblycephala, two SNP sites are found on the cDNA of the gene Egln2 from 10 genes closely associated with hypoxia tolerance (Epo, Hif1α, Hif2α, VhI, Hif1an, Vegfaa, Egln1a, Egln1b, Egln2 and Egln3). Through the association analysis, it has been demonstrated that the two SNP sites of the gene Egln2 are significantly related to the hypoxia tolerance traits of Megalobrama amblycephala. Further, the two diplotype strains are exposed to hypoxia stress, and the results show that there are great differences between the two hypoxia-tolerant diplotype strains in the red blood cell count, hemoglobin (Hb) concentration, protruding length and respiration area of the gill lamella and the ILCM thickness and volume, so the diploid type strain can be used as a molecular marker related to hypoxia-tolerant traits to promote the breeding of a new hypoxia-tolerant Megalobrama amblycephala strain.

### 1. Materials and Methods

### 1.1 Experimental Materials

Hypoxia-tolerant F₅ generation of Megalobrama amblycephala and control Megalobrama amblycephala “Pujiang No. 1”, weighing 40-50 g, were both purchased from the Genetics and Breeding Center of the Ministry of Agriculture of Shanghai Ocean University. The experimental materials were temporarily kept in an indoor plastic aquarium under the following conditions: temperature: 25±1.0° C.; dissolved oxygen: 7.0±0.5 mg/L; pH: 7.5±0.3. ⅓ of the water in the aquarium was replaced with aerated tap water and the feeding was performed twice every day (8:00 a.m. and 5:00 p.m.). The aquarium was kept under natural light.

### 1.2 Enzyme Activity Determination and Genomic DNA Extraction

100 hypoxia-tolerant F₅ generation samples of Megalobrama amblycephala and 100 control Megalobrama amblycephala “Pujiang No. 1” samples were both subjected to acute hypoxia stress under 25±1.0° C. and dissolved oxygen of 2.0 mg/L for 4 h. Then the gill and fin ray of individual Megalobrama amblycephala were collected, where the gill was stored at −80° C. and the fin ray was kept in absolute ethanol.

Each gill tissue was mixed with a 0.86% normal saline in a weight (g)-volume (mL) ratio of 1:9 to prepare a homogenate, which was then tested for the activities of catalase (CAT), superoxide dismutase (SOD) and Na⁺/K⁺-ATPase using a kit from Nanjing Jiancheng Bioengineering Institute.

The genomic DNA was extracted with the help of TIANamp Marine Animals DNA Kit (Tiangen Biotech (Beijing) Co., Ltd), and then tested for the weight and concentration respectively by 1.5% agarose gel electrophoresis and a spectrophotometry. The genomic DNA was stored at −20° C. for use.

### 1.3 Screening of SNP Sites on Gene Egln2

Based on the known transcriptome data associated with hypoxia tolerance in Megalobrama amblycephala, 10 genes closely associated with hypoxia tolerance (Epo, Hif1α, Hif2α, VhI, Hif1an, Vegfaa, Egln1a, Egln1b, Egln2 and Egln3) were selected and respectively compared with the genomic sequence of Megalobrama amblycephala to obtain an intron and an exon of individual gene. Primers were designed according to the intron of each gene using PrimerPremier software to amplify the corresponding exon. The experimental results indicated that there were two differential SNP sites respectively at position 397 and position 715 of the gene Egln2. The sample size was expanded for further demonstration. The sequences of the primers designed herein and the lengths of the amplification products were shown in Table 1, and the entire coding region of the genomic DNA was covered.

TABLE 1
Primer information

| | | Amplification | Annealing |
|---|---|---|---|
| Primer | Sequence (5′-3′) | length/bp | temperature/° C. |
| P1 | GTTTACTGTCGAGCTTCGTGAT (SEQ ID NO: 1) | 696 | 55 |
| | AAGGGGAGCGACAGCCTGTGT (SEQ ID NO: 2) | | |
| P2 | CGGAAATGCCCCATGGACT (SEQ ID NO: 3) | 727 | 55 |
| | TTTAGTGCAATGGAACAGTTTCA (SEQ ID NO: 4) | | |
| P3 | ATTTAACCGTTCATCACCCTT (SEQ ID NO: 5) | 352 | 55 |
| | GACGCTTTTAAGCCTGCCGTT (SEQ ID NO: 6) | | |
| P4 | TCAGTCAAGATGAGAGTCTTCTCT (SEQ ID NO: 7) | 300 | 55 |
| | CAATTGTAGACTTTGAGTCACTGAAT (SEQ ID | | |
| | NO: 8) | | |
| P5 | GCACATCTCAAACTCCATATCA (SEQ ID NO: 9) | 302 | 55 |
| | GAAAATGTGTTCCCACCAACC (SEQ ID NO: 10) | | |
| P6 | AAAGGTCAGGTCATTTTTGCTA (SEQ ID NO: 11) | 334 | 55 |
| | ATGTTGGGCTTCTCGCATCTT (SEQ ID NO: 12) | | |

20 samples from the hypoxia-tolerant F₅ generation of Megalobrama amblycephala and 20 samples from the control Megalobrama amblycephala “Pujiang No. 1” were respectively subjected to PCR amplification using the above 6 pairs of primers, where the PCR system had a total volume of 20 μL and consisted of 10 μL of 2×Taq PCR Mastermix, 1 μL of template DNA (50 ng/μL), 1 μL of forward primer (10 μmol/L), 1 μL of reverse primer (10 μmol/L) and 7.0 μL of ddH₂O; and the PCR amplification was programmed as follows: pre-denaturation at 94° C. for 3 min; 35 cycles and each consists of denaturation at 94° C. for 30 s, annealing at 55° C. for 30 s and extension at 72° C. for 45 s; and extension at 72° C. for 7 min. The amplification products were detected by 1.5% agarose gel electrophoresis, and the valid amplification products were sequenced by Shanghai Map Biotech Co., Ltd. The sequencing results were compared by Sequencher 5 to find SNP sites, and then the primers involving the SNP sites were amplified and verified using 100 hypoxia-tolerant F₅ generation samples of Megalobrama amblycephala and 100 control Megalobrama amblycephala “Pujiang No. 1” samples. The reagents used herein were all purchased from Tiangen Biotech (Beijing) Co., Ltd, and the primer synthesis was completed by Shanghai Map Biotech Co., Ltd.

1.4 Determination of Critical Oxygen Tension (LOE_{crit}) of Diploid Types I and II at which they Lose the Equilibrium

In order to verify the obtained SNP sites and the constructed predominant diploid type, 15 diploid type I strains and 15 diploid type II strains, weighing 40-50 g, were selected and kept in a 20 L glass aquarium for 12 h respectively at 10° C. (DO=10±0.5 mg/L), 25° C. (DO=8.5±0.5 mg/L) and 30° C. (DO=7.5±0.5 mg/L). A gauze having the same size as the water surface was provided 5 cm below the water surface to prevent the Megalobrama amblycephala from surfacing. Nitrogen and air were simultaneously introduced to the glass aquarium, and the dissolved oxygen content in the water was monitored in real time by a dissolved oxygen meter. The dissolved oxygen variation was programmed as follows: decreasing from normoxia (≥10 mg/L) to 5 mg/L in 1 h; 5 mg/L for 1 h; decreasing from 5 mg/L to 2.5 mg/L in 30 min; 2.5 mg/L for 30 min; decreasing from 2.5 mg/L to 1 mg/L in 30 min; 1 mg/L for 30 min; decreasing from 1 mg/L to 0.5 mg/L in 30 min; 0.5 mg/L for 30 min; and decreasing from 0.5 mg/L to 0 mg/L. The experiment at individual temperature was repeated 3 times. When the Megalobrama amblycephala lost the equilibrium to turn on its side, the dissolved oxygen content and the time consumed were recorded. The LOE_{crit} value was calculated according to the Brett's method:

$${LOE}_{crit} = {[ {O}_{2} ]}_{2 i} - {( \frac{{t}_{i}}{{t}_{ii}} ) [ {O}_{2} ]}_{2 ii} ; \qquad ( 1 )$$

where [O₂]₂ᵢ indicated the penultimate dissolved oxygen content of the hypoxic treatment (mg/L); [O₂]₂ᵢᵢ indicated the reduced value of dissolved oxygen in each reduction; tᵢ indicated the time it takes for the experimental Megalobrama amblycephala to lose equilibrium; tᵢᵢ indicated the time of each steady phase, i.e., 0.5 h.

### 1.5 Hypoxia Stress

20 diploid type I strains and 20 diploid type II strains, weighing 40-50 g, were placed in a 20 L liquid bath thermostatic circulation tank (T=10° C.). Nitrogen and air were introduced, and the dissolved oxygen content in the water was monitored by a dissolved oxygen meter and kept below 2.0 mg/L to perform hypoxia stress for 7 days.

### 1.6 Tissue Section

5 diploid type I strains and 5 diploid type II strains were collected respectively after 0 day, 4 days and 7 days of hypoxia stress and after 7 days of recovery, anesthetized with 0.5 g/L MS-222 and subjected to vivisection on ice, where the gill lamellae isolated from the right second gill arch was subsequently observed by an optical microscope. The gill lamellae was repeatedly washed with 0.75% normal saline to remove the blood cells and mucus adhering to the gill filaments and fixed in Boiun's solution for 24 h. Then the gill lamellae was subjected to conventional dehydration and tissue clearing with xylene, embedded with paraffin and subjected to serial sectioning (with a thickness of 5 μm). The sections were subjected to H-E staining, and observed and photographed under a microscope.

### 1.7 Morphological Analysis

In order to evaluate the changes of the lamella respiration area and interlamellar cell mass (ILCM) volume of diploid type I and diploid type II, the following morphological parameters were measured: (1) lamella basal length (l); (2) lamella thickness (t) and (3) protruding lamella length (h). Each parameter was measured 30 times during the same hypoxia treatment time. The gill lamella was approximated as a semi-ellipse and calculated for its area according to the following equations:

$$\begin{matrix}a = p 1 ; & ( 2 ) \\ p = \frac{2 π \sqrt{\frac{1}{2} ( {r}^{2} + {h}^{2} )}}{2} ; & ( 3 ) \\ r = t / 2 ; & ( 4 ) \\ V = {dH}_{i} 1 ; & ( 5 )\end{matrix}$$

where in equation (2), “a” represented the lamella respiration area (μm²); “1” represented the lamella basal length (μm); “p” represented the circumference of the ellipse (μm);

in equation (3), “h” represented the protruding lamella length (μm);

in equation (4), “t” represented the lamella thickness (μm);

in equation (5), “d” represented the distance between adjacent lamellas (μm); and “Hᵢ” represented the ILCM thickness (μm).

### 1.8 Blood Analysis

The weight and length of the Megalobrama amblycephala which lost equilibrium under hypoxia stress were recorded. Then the blood sample was collected from the tail vein with 1 mL plastic syringe pre-washed with heparin and measured for the Hb concentration and the RBC count using standard methods.

### 1.9 Statistical Analysis

The data were all expressed as mean±standard deviation (SD), and the significance analysis was performed using two-way analysis of variance (ANOVA) (Tukey's pair-wise method), where p<0.05 indicated a significant difference, and p<0.01 indicated a very significant difference. The genetic parameters were analyzed using Popgene software. The correlation between individual SNP site on the gene Egln2 and the hypoxia-tolerant traits of the Megalobrama amblycephala was analyzed by chi-square test using JMP 8.0 software. The linkage disequilibrium analysis and haplotype construction were performed on line by SHEsis.

### 2. Results

### 2.1 Amplification of Gene Egln2

Based on the known transcriptome data of Megalobrama amblycephala, differential SNP sites were screened from 10 genes related to hypoxic tolerance traits. It was found that there were differential SNP sites on positions 397 and 715 of the gene Egln2. 6 pairs of primers were designed according to the gene Egln2, and amplified to obtain corresponding gene fragments using 20 samples of hypoxia-tolerant F₅ generation of Megalobrama amblycephala and 20 control Megalobrama amblycephala “Pujiang No. 1” strains. The amplified fragments were sequenced and compared, and one SNP site was found on each of the fragments amplified from the two pairs of primers P1 and P2: T³⁹⁷C³⁹⁷ and T⁷¹⁵G⁷¹⁵ (Table 2). No SNP sites were found on the fragments amplified from the other 4 pairs of primers.

TABLE 2
Genotype and gene frequency of SNP sites in gene Egln2 of
Megalobrama amblycephala

| | Geno- | | Genotype | | Allele |
|---|---|---|---|---|---|
| Locus | type | Number | frequency/% | Allele | frequency/% |
| T³⁹⁷C³⁹⁷ | CC | 31 | 15.5 | C | 38.75 |
| | TC | 93 | 46.5 | T | 61.25 |
| | TT | 76 | 38.0 | | |
| T⁷¹⁵G⁷¹⁵ | GG | 31 | 15.5 | G | 38.75 |
| | TG | 93 | 46.5 | T | 61.25 |
| | TT | 76 | 38.0 | | |

### 2.2 Polymorphism Analysis of Gene Egln2

The SNP sites screened on the Egln2 gene were amplified using 100 samples of hypoxia-tolerant F₅ generation of Megalobrama amblycephala and 100 control Megalobrama amblycephala “Pujiang No. 1” strains, and sequenced. The sequencing results showed that the two SNP sites on the Egln2 gene both underwent a missense mutation, where with respect to the T³⁹⁷C³⁹⁷ locus, the codon was converted from TAC to CAC, and the corresponding amino acid was converted from tyrosine to histidine; with respect to the T⁷¹⁵G⁷¹⁵ locus, the codon was converted from TTG to GTG, and the corresponding amino acid was converted from leucine to valine.

The polymorphic parameters of the SNP locus in the Egln2 gene were presented in Table 3.

According to the principle that PIC (polymorphism information content)>0.5, 0.25<PIC<0.5 and PIC<0.25 respectively indicated high polymorphism, moderate polymorphism and low polymorphism, the two SNP sites in the Egln2 were both of high polymorphism.

TABLE 3
Polymorphic parameters of the SNP locus in the Egln2
gene of Megalobrama amblycephala

| Locus | Ho | He | PIC | Ne |
|---|---|---|---|---|
| T³⁹⁷C³⁹⁷ | 0.465 | 0.476 | 0.668 | 1.904 |
| T⁷¹⁵G⁷¹⁵ | 0.465 | 0.476 | 0.668 | 1.904 |

### 2.3 Association Analysis Between SNP Sites in Egln2 Gene and Hypoxia Traits

Association analysis was performed between the genotype of the SNP sites in Egln2 gene and activities of hypoxia trait-related enzymes (CAT, SOD and Na⁺/K⁺-ATPase) respectively from 100 samples of hypoxia-tolerant F₅ generation of Megalobrama amblycephala and 100 control Megalobrama amblycephala “Pujiang No. 1” strains. It can be seen from Table 4 that there was significant difference in the activities of the hypoxia trait-related enzymes (CAT, SOD and Na⁺/K⁺-ATPase) (p<0.05) among different genotypes of the two SNP sites.

TABLE 4
Association analysis between 2 SNP sites in Egln2 gene and hypoxia
trait-related enzymes

| Locus | Genotype | Number | Na⁺/K⁺-ATPase | CAT | SOD |
|---|---|---|---|---|---|
| T³⁹⁷C³⁹⁷(T⁷¹⁵G⁷¹⁵) | C³⁹⁷C³⁹⁷(G⁷¹⁵G⁷¹⁵) | 31 | 17.218 ± 1.214^{b} | 2.855 ± 0.236^{b} | 31.388 ± 2.103^{a} |
| | T³⁹⁷C³⁹⁷(T⁷¹⁵G⁷¹⁵) | 93 | 20.514 ± 0.701^{a} | 3.362 ± 0.136^{ab} | 30.310 ± 1.214^{a} |
| | T³⁹⁷T³⁹⁷(T⁷¹⁵T⁷¹⁵) | 76 | 20.935 ± 0.775^{a} | 3.592 ± 0.151^{a} | 26.264 ± 1.343^{b} |

Notes:
different letters in the same column indicated significant difference, p < 0.05.

### 2.4 Analysis of Linkage Disequilibrium and Haplotype of SNP Sites on Egln2 Gene

The linkage disequilibrium of the differential sites was analyzed using the SHEsis software, and the results revealed that the two sites T³⁹⁷C³⁹⁷ and T⁷¹⁵G⁷¹⁵ were in complete linkage disequilibrium (D′=1, R²=1), which was consistent with the results of genotype frequencies, gene frequencies and genetic parameters of the two sites in complete linkage (shown in Table 2 and Table 3). The two SNP sites were used in the haplotype construction and analysis, where the haplotype with a frequency less than 1% was removed and a total of 2 haplotypes were obtained. It was found that there was very significant difference (p<0.01) between the occurrence frequencies of haplotype I (C³⁹⁷G⁷¹⁵) in the population of control Megalobrama amblycephala “Pujiang No. 1” (83.5%) and the population of hypoxia-tolerant F₅ generation of Megalobrama amblycephala (27.5%), and the difference between the occurrence frequencies of haplotype II (T³⁹⁷T⁷¹⁵) in the population of control Megalobrama amblycephala “Pujiang No. 1” (16.5%) and the population of hypoxia-tolerant F₅ generation of Megalobrama amblycephala (72.5%) was also very significant (p<0.01, Table 5).

TABLE 5
Haplotype analysisof 2 SNP sites in Egln2 gene

| | | | Hypoxia-tolerant | |
|---|---|---|---|---|
| | | Megalobrama | F₅ generation of | |
| | | amblycephala | Megalobrama | |
| Haplo- | | ″Pujiang No. 1″ | amblycephala | |
| type | Sequence | (frequency) | (frequency) | χ² (p value) |
| I | C³⁹⁷G⁷¹⁵ | 167 (0.835) | 55 (0.275) | 21.330 (3.96 x 10^{-6**}) |
| II | T³⁹⁷T⁷¹⁵ | 33 (0.165) | 145 (0.725) | 21.330 (3.96 x 10^{6**}) |

Notes:
^{**}indicated extremely significant difference (p < 0.01).

### 2.5 Diploid Type Analysis of 2 SNP Sites in Egln2 Gene

The two haplotypes were randomly combined into a diploid type, where the combination with an occurrence rate less than 5% was removed and a total of three diploid types were obtained, namely diploid type I (C⁹⁷C⁹⁷G⁷¹⁵G⁷¹⁵) and diploid type II (T^{397T3}T⁹⁷¹⁵T⁷¹⁵) and diploid type III (T⁹⁷C³⁹⁷T⁷¹⁵G⁷¹⁵). The correlation between individual diploid type and the hypoxia tolerance-related traits was analyzed, and the results showed that the activities of hypoxia trait-related enzymes in diploid type II were significantly higher than those in the diploid type I and diploid type III (p<0.05, Table 6).

TABLE 6
Association analysis between diploid type of the SNP sites in Egln2
gene and activities of hypoxia trait-related enzymes

| | Haploid | | | |
|---|---|---|---|---|
| Diploid type | type | Na⁺K⁺-ATP | CAT | SOD |
| Diploid type I | I/I | 17.22 ± 4.35^{b} | 2.86 ± 1.04^{b} | 26.39 ± 1.42^{b} |
| (C³⁹⁷C³⁹⁷G⁷¹⁵G⁷¹⁵) | | | | |
| Diploid type II | II/II | 28.93 ± 1.68^{a} | 6.59 ± 1.50^{a} | 38.26 ± 2.56^{a} |
| (T³⁹⁷T³⁹⁷T⁷¹⁵T⁷¹⁵) | | | | |
| Diploid type III | I/II | 19.32 ± 2.05^{b} | 3.06 ± 1.22^{b} | 27.12 ± 2.60^{b} |
| (T³⁹⁷C³⁹⁷T⁷¹⁵G⁷¹⁵) | | | | |

Notes: different letters in the same column indicated significant difference (p <0.05).

2.6 Critical Oxygen Tension at which the Megalobrama amblycephala Loses the Equilibrium

The critical oxygen tension (LOE_{crit}) at which the fish lost the equilibrium was affected by temperature. Specifically, the diploid type I strain had a LOE_{crit} value of 0.69 mg/L at 10° C., 0.90 mg/L at 25° C. and 1.31 mg/L at 30° C., which indicated that the hypoxia tolerance of the diploid type I strain was significantly lowered with the increase in temperature (p<0.01); and the diploid type II strain had a LOE_{crit} value of 0.46 mg/L at 10° C., 0.81 mg/L at 25° C. and 1.18 mg/L at 30° C., which also showed significant difference in hypoxia tolerance among different temperatures (p<0.01).

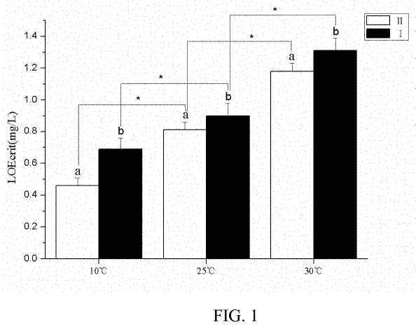

At the same temperature, the LOE_{crit} value of the diploid type II strain was higher than that of the diploid type I strain. It can be seen from FIG. 1 that with regard to the hypoxia tolerance, the diploid type II strain was 50% higher than the diploid type I strain at 10° C. (the LOE_{crit} values of the diploid type II strain and the diploid type I strain were 0.46 mg/L and 0.69 mg/L, respectively), 11.1% higher than the diploid type I strain at 25° C. (the LOE_{crit} values of the diploid type II strain and the diploid type I strain were 0.81 mg/L and 0.90 mg/L, respectively) and 11.0% higher than the diploid type I strain at 30° C. (the LOE_{crit} values of the diploid type II strain and the diploid type I strain were 1.18 mg/L and 1.31 mg/L, respectively).

### 2.7 Change in Gill Morphology

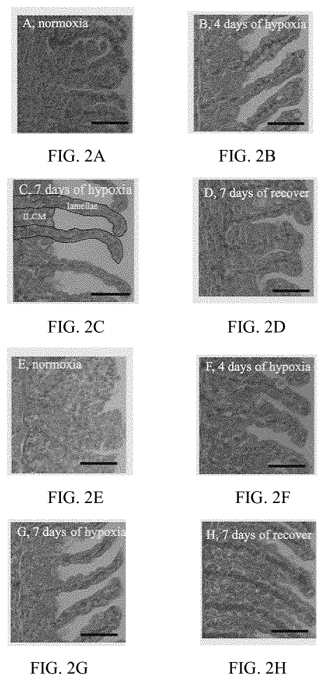

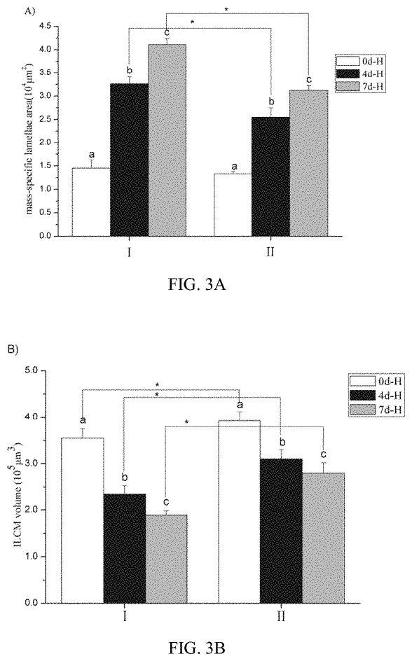

Under hypoxia conditions (temperature: 10±1.0° C. and DO: 2.0 mg/L), the diploid type II strain and the diploid type I strain both experienced continuous increase in the respiratory area. As shown in FIGS. 2A-2H and FIGS. 3A-3B, after exposed to hypoxia for 7 days, the respiratory area of the diploid type I strain (4.11×10⁴ μm²) was significantly larger than that of the diploid type II strain (3.12×10⁴ μm²) (p<0.01).

After exposed to hypoxia conditions (temperature: 10±1.0° C. and DO: 2.0 mg/L) for 4 days and 7 days, the diploid type II strain had an ILCM volume respectively of 3.1×10⁵ μm³ and 2.8×10⁵ μm³, and the diploid type I strain had an ILCM volume respectively of 2.35×10⁵ μm³ and 1.89×10⁵ μm³, which indicated that both of the diploid type II strain and the diploid type I strain underwent a rapid decrease in the ILCM volume with the extension of the hypoxia treatment. However, through the same treatment time, the ILCM volume of the diploid type I strain was significantly less than that of the diploid type II strain (p<0.01), which indicated that the diploid type II strain has better hypoxia tolerance (FIGS. 2A-2H and FIGS. 3A-3B).

### 2.8 Changes in the RBC Count and Hb Concentration

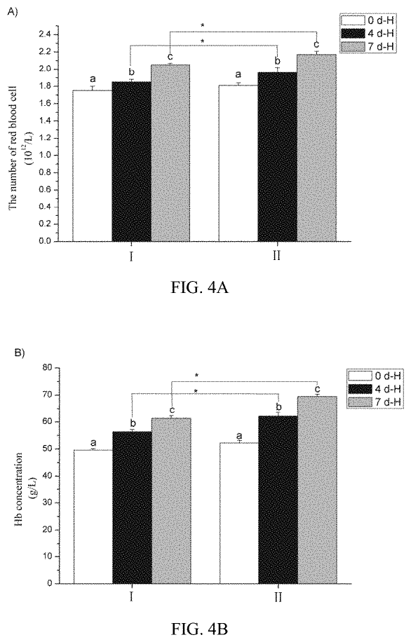

Under normoxia, the average RBC count of the diploid type I strain was 1.75×10¹²/L, and after exposed to hypoxia for 4 days and 7 days, the RBC count was 1.85×10¹²/L and 2.05×10¹²/L, respectively, which indicated that the RBC count in the diploid type I strain was significantly increased (p<0.01, FIG. 4A). Under normoxia, the Hb concentration of the diploid type I strain was 49.6±2.1 g/L, and after exposed to hypoxia for 4 days and 7 days, the Hb concentration was significantly increased to 56.3±0.9 g/L and 61.3±1.0 g/L, respectively (p<0.01, FIG. 4B).

After exposed to hypoxia for 4 days and 7 days, the average RBC count in the diploid type II strain was 1.96×10¹²/L and 2.17×10¹²/L, respectively, which were significantly higher than those of the diploid type I strain (p<0.01, FIG. 4A). Moreover, after exposed to hypoxia for 4 days and 7 days, the Hb concentration of the diploid type II strain was significantly increased to 62.1±0.9 g/L and 69.5±0.8 g/L from 52.2±0.9 g/L (under normoxia), respectively (p<0.01, FIG. 4B). Whether after the 7-day exposure or 4-day exposure to hypoxia, the Hb concentration of the diploid type II strain was significantly higher than that of the diploid type I strain (p<0.01, FIG. 4B).

As a heritable trait, the fish hypoxia tolerance is a quantitative trait controlled by multiple genes. Prolyl hydroxylase, as a rate-limiting enzyme related to hypoxia in organisms, plays an important role in the process of hypoxia response. Primers used herein were designed according to the cDNA sequence of the isolated Egln2 gene of Megalobrama amblycephala, amplified and subjected to sequence comparison, and two SNP sites were found on the cDNA sequence of Egln2 gene, namely T³⁹⁷C³⁹⁷ and T⁷¹⁵G⁷¹⁵. The polymorphism analysis results showed that the two sites in the Egln2 gene both exhibited high polymorphism. Further, it was demonstrated through the linkage disequilibrium analysis that the sites T³⁹⁷C³⁹⁷ and T⁷¹⁵G⁷¹⁵ were in complete linkage disequilibrium. The linkage of multiple sites may contain more information and has better prospect in the molecular marker-assisted breeding.

Among the measured parameters related to hypoxia tolerance, superoxide dismutase (SOD) and catalase (CAT) played a key role in the biological antioxidant system, and the Na⁺/K⁺-ATPase is a key enzyme involved in energy metabolism, which all can be used as an indicator to evaluate the hypoxia tolerance of fish. In this application, when 100 samples of hypoxia-tolerant F₅ generation of Megalobrama amblycephala and 100 control Megalobrama amblycephala “Pujiang No. 1” strains were used to detect the 2 SNP sites on the Egln2 gene, it was found that different genotypes of these 2 sites showed significant difference in the activities of SOD, CAT and Na⁺/K⁺-ATPase (p<0.05), which demonstrated that the Egln2 gene was closely associated with the hypoxia tolerance of Megalobrama amblycephala. The breeding, which was conducted on the genotypes at different sites on the Egln2 gene, can not only improve the hypoxia tolerance of Megalobrama amblycephala, but also promote the breeding of new hypoxia-tolerant Megalobrama amblycephala strains. Both of the two SNP sites in the Egln2 gene experienced a missense mutation, and these two sites were located in the coding region of the gene and were significantly associated with hypoxia trait-related enzymes (SOD, CAT and Na⁺/K⁺-ATPase), which may be explained by that the missense mutation led to the change in the coded amino acid, further changing the activity of prolyl hydroxylase and affecting the hypoxia tolerance-related traits.

Some problems, for example, the information of some loci was vague and incomplete, can not be avoided in the previous analysis of the correlation between single SNP site and target trait using a traditional method, and these problems can be effectively overcome through the construction and analysis of haplotype. This application selected SNP sites on the Egln2 gene that were significantly different in the activities of hypoxia trait-related enzymes (SOD, CAT and Na⁺/K⁺-ATPase), and further employed these sites for the construction and analysis of haplotype. In the two haplotypes constructed based on the Egln2 gene, the haplotype I was predominant in the population of Megalobrama amblycephala “Pujiang No. 1”, and the haplotype II was predominant in the population of hypoxia-tolerant F₅ generation of Megalobrama amblycephala. The occurrence frequency of the haplotype I in the population of Megalobrama amblycephala “Pujiang No. 1” was significantly higher than that in the population of hypoxia-tolerant F₅ generation of Megalobrama amblycephala, conversely, the occurrence frequency of the haplotype II in the population of hypoxia-tolerant F₅ generation of Megalobrama amblycephala was significantly higher than that in the population of Megalobrama amblycephala “Pujiang No. 1”. Compared to other combinations, the diploid type II had significantly higher activities of hypoxia trait-related enzymes (SOD, CAT and Na⁺/K⁺-ATPase) (p<0.05), which indicated that the homozygous mutations at these two sites were advantageous mutations for the hypoxia tolerance. Given the above, the haplotype I and haplotype II can be used as predominant haplotype in the population of Megalobrama amblycephala “Pujiang No. 1” and in the population of hypoxia-tolerant F₅ generation of Megalobrama amblycephala, respectively. A strain with predominant genotype can be selected to promote the breeding of a new hypoxia-tolerant Megalobrama amblycephala strain.

LOE_{crit} value was used as an index to evaluate the hypoxia tolerance of fish. Fish with lower LOE_{crit} value can adapt to lower dissolved oxygen content better. In this application, the diploid type II strain had a LOE_{crit} value of 0.81±0.05 mg/L at 25° C., which was much lower than the LOE_{crit} value of some marine fish. The diploid type I strain and the diploid type II strain had a LOE_{crit} value of 0.72 mg/L and 0.54 mg/L at 10° C., respectively. Meanwhile, the diploid type I strain had an LOE_{crit} value of 1.03 mg/L and 1.41 mg/L respectively at 25° C. and 30° C. At the same temperature, the LOE_{crit} value of the diploid type I strain was higher than that of the diploid type II strain, which demonstrated that the diploid type II strain was superior to the diploid type I strain in the hypoxia tolerance.

The dissolved oxygen content was associated with the environmental conditions. In order to adapt to changes in dissolved oxygen, fish either increased the respiration surface area of gill or increase the oxygen carrying capacity of the blood. It has been found before that Megalobrama amblycephala had the ability to remodel its gill in a low-oxygen water environment. In this application, the diploid type I strain and the diploid type II strain both experienced increase in the lamella area and decrease in the ILCM volume under hypoxic condition. In natural water, the lamella area was reduced due to the proliferation of ILCM cells, which was not conducive to the fish respiration. It was observed herein that after exposed to hypoxia stress at 10° C. for 7 days, the diploid type II strain was significantly lower than the diploid type I strain in the protruding lamella length and the lamella area (p<0.01). The above results revealed that the diploid type II strain had better potential to prevent the toxic substances from flowing into the blood and further cope with hypoxic stress.

The pervious transcriptome analysis of Megalobrama amblycephala revealed that the mRNA of erythropoietin was significantly up-regulated under hypoxic conditions, which can further cause an increase in the RBC count and the Hb concentration, improving the oxygen-carrying capacity of the blood. As disclosed herein, after exposed to hypoxia for 4 days and 7 days, the diploid type II strain was significantly higher than the diploid type I strain in the RBC count and the Hb concentration (p<0.01). The extremely high hemoglobin-oxygen affinity can ensure there was enough oxygen in the blood even if the gill was completely surrounded by cell clusters, which may be the reason why the ILCM volume of the diploid type II strain was smaller than that of the diploid type I strain on the 4′ and 7′ day of hypoxia.

In summary, the diploid type II strain exhibited higher hypoxia tolerance, and the haplotype II can be used in the molecular marker-assisted breeding of a new hypoxia-tolerant Megalobrama amblycephala strain.

Described above are merely preferred embodiments of the invention. It should be understood that any modifications, replacements and changes made by those skilled in the art without departing from the spirit of the invention should fall within the scope of the invention.

What is claimed is:

1. A method of breeding a hypoxia-tolerant Megalobrama amblycephala strain, comprising:
(1) screening genes associated with hypoxia tolerance in Megalobrama amblycephala based on transcriptome data;
(2) screening SNP sites from the genes associated with hypoxia tolerance; and
(3) applying a haplotype of the SNP sites in the molecular marker-assisted breeding of Megalobrama amblycephala to obtain the hypoxia-tolerant Megalobrama amblycephala strain.

2. The method of claim 1, wherein the step (2) comprises:
comparing a sequence of each of the genes associated with hypoxia tolerance with genome sequence of Megalobrama amblycephala to obtain an intron and an exon of each of the genes associated with hypoxia tolerance;
designing a pair of primers within the intron of each of the genes associated with hypoxia tolerance to amplify the corresponding exon; and
comparing a sequence of the amplified product with a sequence of the corresponding exon in genome of Megalobrama amblycephala to screen the SNP sites;
wherein the SNP sites have a polymorphism information content greater than 0.5.

3. The method of claim 2, wherein the genes associated with hypoxia tolerance comprise Epo, Hif1α, Hif2α, VhI, Hif1an, Vegfaa, Egln1a, Egln1b, Egln2 and Egln3.

4. The method of claim 1, wherein the SNP sites are located at positions 397 and 715 of gene Egln2; and the haplotype of the SNP sites is T³⁹⁷T⁷¹⁵.

5. The method of claim 2, wherein the SNP sites are located at positions 397 and 715 of gene Egln2; and the haplotype of the SNP sites is T³⁹⁷T⁷¹⁵.
